SEQUENCE LISTING (4 sequences)

SEQ ID NO: 1 - protein, 246 aa, Streptococcus
MKIFVLNAQHDEAVDANFDQFNKYGVSDYYKNLINNAKTVEGVKDLQAQVVESAKKARISEATDGLSDFLQSQTPAEDTVKSIELAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPKTDTYKLILNGKTLKGETTTEAVDAATARSFNFPILENSRG

SEQ ID NO: 2 - protein, 3 aa, Artificial sequence
  Chemically synthesized support peptide
MEF

SEQ ID NO: 3 - protein, 101 aa, Respiratory syncytial virus
TVKTKNTTTTQTQPSKPTTKQRQNKPPNKPNNDFHFEVFNFVPCSICSNNPTCWAICKRIPNKKPGKKTTTKPTKKPTFKTTKKDHKPQTTKPKEVPTTKP

SEQ ID NO: 4 - protein, 104 aa, Artificial sequence
  Chemically synthesized MEFG2Na peptide
MEFTVKTKNTTTTQTQPSKPTTKQRQNKPPNKPNNDFHFEVFNFVPCSICSNNPTCWAICKRIPNKKPGKKTTTKPTKKPTFKTTKKDHKPQTTKPKEVPTTKP

(19) United States
(12) Patent Application Publication
(10) Pub. No.: US 2008/0300382 A1
(43) Pub. Date: Dec. 4, 2008

(54) Immunogenic Complexes, Preparation Method Thereof And Use Of Same In Pharmaceutical Compositions

(75) Inventors: Christine Libon, St. Julien en Genevois (FR); Thien Nguyen, Tolosan (FR)

Correspondence Address: BIRCH STEWART KOLASCH & BIRCH, PO BOX 747, FALLS CHURCH, VA, 22040-0747, US

(73) Assignee: PIERRE FABRE MEDICAMENT, Boulogne-Billancourt (FR)

(21) Appl. No.: 11/658,173

(22) PCT Filed: Jul. 25, 2005
(86) PCT No.: PCT/FR2005/001913

§ 371 Date: Jan. 23, 2007

(30) Foreign Application Priority Data
Jul. 23, 2004 (FR) 0408175

(51) Int. Cl. C07K 2/00; C07K 1/107; C07K 14/135; C07H 21/04; C07K 5/083

(52) U.S. Cl. 530/322; 530/345; 530/331; 536/23.72; 536/23.1

(57) ABSTRACT

The invention relates to a method of improving the immunogenicity of an immunogen, antigen or hapten, by means of coupling with a small support peptide. More specifically, the invention relates to a method of preparing an immunogenic complex, as well as the complexes that can be obtained using one such method, and to the use of said complexes as a medicament in order to increase the immunogenicity of an immunogen. The invention comprises, for example, a support peptide which is coupled with a peptide from protein G of the respiratory syncytial virus (RSV) and to the use thereof as a vaccine for the treatment of respiratory infections linked to RSV.

The present invention relates to a method of improving the immunogenicity of an immunogen, antigen or hapten, by means of coupling with a small support peptide. More specifically, the present invention relates to a method of preparing an immunogenic complex, as well as the complexes that can be obtained by one such method, and the use of said complexes as a drug in order to increase the immunogenicity of an immunogen. The invention comprises, for example, a support peptide which is coupled with a peptide from the respiratory syncytial virus (RSV) G protein and the use thereof as a vaccine for the treatment of RSV-related respiratory infections.

The immune system is a network of interacting humoral and cellular components which allows the host to differentiate self molecules from non-self molecules in order to eliminate the latter as well as pathogens. To this end, the immune system has developed two mechanisms which act in concert, namely natural immunity and acquired immunity.

Natural immunity encompasses the physical barriers (skin, mucosa, etc.), cells (monocytes/macrophages, granulocytes, NK cells, etc.) and soluble factors (complements, cytokines, acute phase proteins, etc.) activated or produced in response to an attack. Natural immunity responses are rapid but are neither specific nor memorized.

The cellular mediators of acquired immunity are the T and B lymphocytes. In particular, by their interaction the latter produce immunoglobulins. In contrast with natural immunity responses, those of acquired immunity are specific, adaptable and can be memorized. Indeed, the initial penetration of an antigen into a naïve organism leads to an immune response, known as the primary response, during which long-lived lymphocytes (T and B), called memory cells, multiply. Through these cells, during a second penetration of the same antigen, the immune reaction, known as the secondary reaction, will be faster and more intense. For a primary response to take place, the antigen must first be captured and prepared by antigen-presenting cells, to be presented to the T lymphocytes.

The goal of vaccines is to protect the host by preventing or limiting pathogen invasion. All of the vaccines marketed today fulfill this role by causing antibodies to be produced.

When the vaccinating antigen alone is not able to trigger an immune response, or if an immune response is produced but is too weak, its physical association with a so-called carrier protein possessing T epitopes capable of interacting with T lymphocytes can trigger the desired response. The most commonly known vaccine carrier proteins are the diphtheria and tetanus toxoids.

Among these carrier proteins, the so-called “BB” protein fragment of the G protein of Streptococcus, which is capable of binding albumin and which is the fragment corresponding to residues 24 to 242 of sequence SEQ ID NO 1, can also be cited. This protein can trigger a primary antibody response that is earlier and more intense with respect to the vaccinating antigen associated therewith (Libon et al., Vaccine, 17(5):406-41, 1999). In this context, international patent application WO 96/14416 can also be consulted.

The aim of the present invention is to provide an alternative to carrier proteins which will, as will be seen in the description below, remedy all of the disadvantages related to the use of such carrier proteins. More specifically, the present invention makes it possible to limit the side effects related to the presence of a relatively large carrier protein while allowing high production yields to be achieved.

For purposes of clarity, the advantages of the present invention will be demonstrated in comparison with a state of the art carrier protein, namely the BB carrier protein.

Quite unexpectedly, and contrary to the current body of knowledge accepted by those skilled in the art, the inventors have demonstrated an alternative to the use of carrier proteins. More specifically, the inventors have characterized a method for improving the immunogenicity of an immunogen based on the identification of a peptide, hereafter referred to as a support peptide, of very small size and consequently nonimmunogenic, that facilitates the synthesis thereof and/or the synthesis of the immunogen-support peptide complexes wherein they participate.

For this purpose, the present invention relates to a method of preparing an immunogenic complex in which an immunogen, antigen or hapten is coupled with a support peptide to form the aforesaid immunogenic complex, wherein the aforesaid support peptide consists of a peptide of less than 10 amino acids comprising at least the 3 amino acid residue peptide fragment of sequence SEQ ID NO 2 (Met-Glu-Phe).

The term “immunogen” includes any substance capable of causing an immune response. As a non-limiting example, the immunogen is preferably a protein, a glycoprotein, a lipopeptide or any immunogenic compound comprising in its structure a peptide of at least 5 amino acids, preferably of at least 10, 15, 20, 25, 30 or 50 amino acids, the compound being capable of causing an immune response, notably capable of inducing the production of specific antibodies directed against said peptide, after the administration thereof in a mammal.

In the present description, the terms “polypeptides,” “polypeptide sequences,” “peptides” and “proteins” are interchangeable.

With respect to the description above, it should be clearly understood that the expression “support peptide” is not the equivalent of the expression “carrier protein.” Indeed, a carrier protein is characterized by its large size (218 amino acids for BB protein) and above all by the presence of T epitopes capable of binding to the T antigen receptors on the surface of the T lymphocytes. The support peptide according to the present invention differs from a carrier protein due to the fact that the support peptide is much smaller (less than 10 amino acids) and the fact that the support peptide does not exhibit T epitopes.

According to a first advantageous aspect, the method according to the present invention makes it possible to produce immunological complexes that improve the immunogenicity of an immunogen for which production is easier or for which production yields are higher. Indeed, the complex comprising the support peptide according to the present invention being much smaller than complexes comprising carrier proteins of the prior art, said support peptide complex is easier to produce by peptide/chemical synthesis or any other technique known to those skilled in the art.

According to a second advantageous aspect, the immunogenic complexes according to the invention make it possible to eliminate, at the very least to limit, the adverse effects related to the very nature of the carrier protein. It is accepted by those skilled in the art that a relatively large carrier protein, such as BB, is highly likely to be the cause of undesired immune responses. For example, it has been shown for the tetanus toxoid that prior sensitization of the host to this carrier protein can prevent the development of an antibody response against the antigen associated with the tetanus toxoid during vaccination with a conjugate (Kaliyaperumal et al., Eur. J. Immunol., 25(12):3375-80, 1995). This phenomenon is known as epitopic suppression.

As a consequence, it is clear from the present description that the invention provides an advantageous alternative to the use of carrier proteins. Indeed, due to its small size, the support peptide has no, or very little, chance of being at the origin of side effects or undesirable effects.

According to a preferred embodiment of the present invention, the support peptide of less than 10 amino acids comprises at least the peptide coded by SEQ ID NO 2 and consists of at most 8 amino acids, preferentially at most 5 amino acids, and still better 4 amino acids.

According to another preferred embodiment, the support peptide of less than 10 amino acids according to the present invention consists of the peptide of sequence SEQ ID NO 2.

The association between the aforesaid support peptide and the immunogen can be carried out by any coupling technique known to those skilled in the art that preserves the integrity as well as the immunogenic properties of the immunogen. More specifically, the method according to the invention is characterized in that the aforesaid association consists of covalent coupling. The term “covalent coupling” comprises chemical coupling or protein fusion by the so-called recombinant DNA technique in which the fusion protein is obtained after translation of a nucleic acid coding for the fusion protein (immunogenic complex) by a host cell (eukaryote or prokaryote) transformed with the aforesaid nucleic acid.

The aforesaid support peptide can be coupled at the N-terminal or C-terminal end of the aforesaid immunogen when the aforesaid immunogen is a peptide. Preferably the aforesaid support peptide is coupled at the N-terminal end of the aforesaid immunogen.

The complex between the support peptide and the compound whose immunogenicity is sought to be improved can be produced by recombinant DNA techniques, notably by the insertion or fusion of the DNA coding for the immunogen into the DNA molecule coding for the support.

According to another embodiment, the covalent coupling between the support peptide and the immunogen is carried out by the chemical route according to techniques known to those skilled in the art.

The invention also has as an object a method in which the aforesaid immunogenic complex is obtained by genetic recombination (recombinant protein) using a nucleic acid resulting from the DNA molecule coding for the support peptide fusing with (or inserting into) the DNA coding for the immunogen, if necessary with a promoter.

In this method, a vector containing one such fusion nucleic acid can be used, the aforesaid vector notably having as its origin a DNA vector from a plasmid, a bacteriophage, a virus and/or a cosmid, and the fusion nucleic acid coding for the aforesaid complex can be integrated in the genome of a host cell to be expressed therein.

Thus the method according to the invention comprises, in one of its embodiments, a step of the production of the complex, by genetic engineering, in a host cell.

The host cell can be prokaryotic and in particular can be selected from the group comprising E. coli, Bacillus, Lactobacillus, Staphylococcus and Streptococcus; it can also be a yeast.

According to another aspect, the host cell is a eukaryotic cell, such as a mammalian cell or an insect cell (Sf9).

The fusion nucleic acid coding for the immunogenic complex notably can be introduced into the host cell via a viral vector.

The immunogen used preferably comes from the bacteria, parasites, viruses or antigens associated with tumors, such as the antigens associated with melanomas or derived from beta hCG.

The method according to the invention is particularly suitable for a surface polypeptide of a pathogen. When the aforesaid polypeptide is expressed in the form of a fusion protein, by recombinant DNA techniques, the fusion protein is advantageously expressed, anchored and exposed on the host cell's membrane surface. Nucleic acid molecules are used that are capable of directing antigen synthesis in the host cell.

Said molecules are comprised of a promoter sequence, a secretion signal sequence linked in a functional manner and a sequence coding for a membrane anchoring region, all of which will be adapted by those skilled in the art.

The immunogen notably can be derived from a human RSV type A or B or bovine RSV surface glycoprotein, notably selected from among the F and G proteins.

Particularly advantageous results are obtained with fragments of the human RSV G protein, sub-groups A or B, or bovine RSV.

In a preferred manner, the immunogen consists of a polypeptide coded by the sequence between residues 130 and 230 of the RSV G protein peptide sequence or by any sequence with at least 80% identity with the aforesaid peptide sequence, preferably 85%, 90%, 95% or 98% identity with the sequence between residues 130 and 230 of the peptide sequence of the aforesaid G protein, or a fragment thereof of at least 10 consecutive amino acids, preferably at least 15, 20, 25, 30 or 50 amino acids, capable of inducing the production of specific antibodies directed against said fragment after the administration thereof in a mammal.

In the context of the present invention, “percent identity” or “percent homology” (the two expressions being used interchangeably in the present description) between two nucleic acid or amino acid sequences means the percentage of nucleotides or amino acid residues that are identical between the two sequences to be compared, obtained after the best alignment (optimal alignment), this percentage being purely statistical and the differences between the two sequences being distributed randomly over their length.

Comparisons of sequences between two nucleic acid or amino acid sequences are typically carried out by comparing these sequences after aligning them optimally, the aforesaid comparison being performed by segment or by a “comparison window.” The optimal alignment of sequences for comparison can be performed manually or by means of the Smith-Waterman local homology algorithm (1981) [Ad. App. Math. 2:482], the Needleman-Wunsch local homology algorithm (1970) [J. Mol. Biol. 48:443], the Pearson and Lipman similarity search method (1988) [Proc. Natl. Acad. Sci. USA 85:2444] or by means of computer software using these algorithms (GAP, BESTFIT, FASTA and TFASTA in the Wisconsin Genetics Software Package, Genetics Computer Group, 575 Science Dr., Madison, Wis., or BLAST N or BLAST P comparison software).

The percent identity between two nucleic acid or amino acid sequences is determined by comparing these two aligned sequences optimally in which the nucleic acid or amino acid sequence to be compared can include additions or deletions compared to the reference sequence for an optimal alignment between these two sequences. Percent identity is calculated by determining the number of identical positions for which the nucleotide or the amino acid residue is identical between the two sequences, dividing this number of identical positions by the total number of positions in the comparison window and multiplying the result by 100 to obtain the percent identity between these two sequences.

For example, the BLAST program, “BLAST 2 sequences” (Tatusova et al., “Blast 2 sequences-a new tool for comparing protein and nucleotide sequences,” FEMS Microbiol Lett. 174:247-250) available on the site http://www.ncbi.nlm.nih.gov/gorf/b12.html can be used, the parameters used being the default parameters (in particular for the parameters “open gap penalty”: 5, and “extension gap penalty”: 2; the matrix chosen being for example the matrix “BLOSUM 62” proposed by the program), the percent identity between the two sequences to be compared being calculated directly by the program.

For the sequence of amino acids with at least 80%, preferably 85%, 90%, 95% and 98% identity with a reference sequence of amino acids, those with certain modifications compared to the reference sequence are preferred, in particular a deletion, an addition or a substitution of at least one amino acid, a truncation or an extension. In the case of a substitution of one or more consecutive or nonconsecutive amino acids, substitutions are preferred in which the substituted amino acids are replaced by “equivalent” amino acids. The expression “equivalent amino acids” designates here any amino acid likely to be substituted for one of the amino acids of the base structure without however essentially modifying the biological activities of the corresponding antibodies. These equivalent amino acids can be determined based on their structural homology with the amino acids for which they are substituted, or based on the results of comparative tests of biological activity between the various antibodies likely to be produced.

According to still another preferred embodiment, the method according to the invention is characterized by the fact that the immunogen is the polypeptide of sequence SEQ ID NO 3, or of a sequence having at least 80% identity with sequence SEQ ID NO 3, preferably 85%, 90%, 95% or 98% identity with the sequence between residues 130 and 230 of the peptide sequence of the aforesaid G protein, or one of the fragments of sequence SEQ ID NO 3 of at least 10 consecutive amino acids, preferably of at least 15, 20, 25, 30 or 50 amino acids, capable of inducing the production of specific antibodies directed against said fragment after the administration thereof in a mammal.

The other immunogens suitable for the implementation of the method according to the invention include a derivative of the hepatitis A, B and C virus surface protein, a measles virus surface protein, a parainfluenza virus surface protein, in particular a surface glycoprotein such as hemagglutinin, neuraminidase, hemagglutinin-neuraminidase (HN) and the fusion (F) protein.

According to another embodiment, the present invention relates to a immunogenic complex obtained by the implementation of the method according to the invention.

More specifically, the present invention also has as an object an immunogenic complex comprising an immunogen, antigen or hapten, wherein the aforesaid immunogen is associated with a support peptide of less than 10 amino acids comprising at least the 3 amino acid residue peptide fragment of sequence SEQ ID NO 2.

Preferably, in the aforesaid immunogenic complex according to the invention, the aforesaid support peptide comprising at least the peptide coded by SEQ ID NO 2 consists of at most 8 amino acids, preferentially of at most 5 amino acids, and still better of 4 amino acids.

According to a preferred embodiment, the aforesaid support peptide of the immunogenic complex according to the invention consists of the peptide coded by SEQ ID NO 2.

According to a preferred embodiment, the aforesaid support peptide of the immunogenic complex according to the invention is characterized in that the aforesaid association consists of a covalent coupling between the aforesaid peptide support and the aforesaid immunogen.

According to a preferred embodiment, the aforesaid immunogenic complex according to the invention is characterized in that the aforesaid support peptide is coupled at the N- or C-terminal end of the aforesaid immunogen when the aforesaid immunogen is a peptide, preferably the N-terminal end.

According to a preferred embodiment, the aforesaid immunogenic complex according to the invention is characterized in that the immunogen is an antigen arising from bacteria, parasites and/or viruses.

According a preferred embodiment, the aforesaid immunogenic complex according to the invention is characterized in that the immunogen is a surface protein or glycoprotein, in particular F or G, of the respiratory syncytial virus (RSV), or of a sequence having at least 80% identity with the sequence of the aforesaid F or G protein, preferably 85%, 90%, 95% or 98% identity with the sequence of the aforesaid F or G protein, or a fragment thereof of at least 10 consecutive amino acids, preferably of at least 15, 20, 25, 30 or 50 amino acids, capable of inducing the production of specific antibodies directed against said fragment after the administration thereof in a mammal.

According to a preferred embodiment, the aforesaid immunogenic complex according to the invention is characterized in that the immunogen is the human RSV type A or B G protein or the bovine RSV G protein.

According to a preferred embodiment, the aforesaid immunogenic complex according to the invention is characterized in that the immunogen is the polypeptide of the sequence between residues 130 and 230 of the RSV G protein, ends included, or of a sequence having at least 80% identity with the aforesaid sequence between 130 and 230, or a fragment thereof of at least 10 amino acids of the aforesaid sequence between 130 and 230 of the RSV G protein.

Preferably, the immunogen of the aforesaid immunogenic complex according to the invention is the polypeptide of sequence SEQ ID NO 3. According to still another preferred embodiment, the complex according to the invention is the MEFG2Na complex of sequence SEQ ID NO 4, or an analogous immunogenic complex whose sequence has the MEF sequence of sequence SEQ ID NO 2 in position 1 to 3 followed by:
either a sequence having at least 80% identity with sequence SEQ ID NO 3, preferably 85%, 90%, 95% or 98% identity with sequence SEQ ID NO 3;
or a sequence of a fragment of sequence SEQ ID NO 3 of at least 10 consecutive amino acids, preferably of at least 15, 20, 25, 30 or 50 amino acids, capable of inducing the production of specific antibodies directed against said fragment after the administration thereof in a mammal.

In another aspect, the present invention has as an object a nucleic acid, preferably isolated and/or purified, coding for the immunogenic complexes according to the invention, notably for the MEFG2Na immunogenic complex of sequence SEQ ID NO 4.

The terms “nucleic acid”, “nucleic sequence”, “nucleic acid sequence”, “polynucleotide”, “oligonucleotide”, “polynucleotide sequence” and “nucleotide sequence”, which are used interchangeably in the present description, indicate a specific sequence of nucleotides, modified or not, that define a fragment or a region of a nucleic acid, containing unnatural nucleotides or not, and corresponding to a double-stranded DNA, a single-stranded DNA or to the transcription products of the aforesaid DNAs.

In still another aspect, the present invention has as an object immunogenic complexes according to the invention or nucleic acids coding for the immunogenic complexes according to the invention used as a drug, notably the MEFG2Na immunogenic complex of sequence SEQ ID NO 4 or the nucleic acid, such as a DNA or RNA, coding for said MEFG2Na complex.

Pharmaceutical compositions comprised of the immunogenic complexes according to the invention or such as previously defined, or a nucleic acid, RNA or DNA, coding for such immunogenic complexes, associated with physiologically acceptable excipients, are also objects of the invention. Said compositions are particularly suitable for the preparation of a vaccine.

Immunization could be obtained by the administration of the aforesaid polynucleotide coding for the immunogenic complexes such as previously defined, alone or via a viral vector comprising one such polynucleotide. A host cell can also be used, notably a killed bacterium, transformed with one such polynucleotide according to the invention.

The present invention also has an object the use of an immunogenic complex according to the invention, in which the aforesaid immunogen complex is a protein or a peptide derived from the RSV G or F protein such as previously defined, notably the MEFG2Na complex or one of its analogues according to the invention, or a nucleic acid according to the invention coding for the aforesaid immunogenic complex, for the preparation of a pharmaceutical composition intended for the prevention or treatment of RSV-related respiratory infections.

The advantages of the present invention will be demonstrated by virtue of the examples and figures below in which:

FIG. 1 represents the anti-RSV-A IgG concentration in mice immunized with BBG2Na or MEFG2Na;

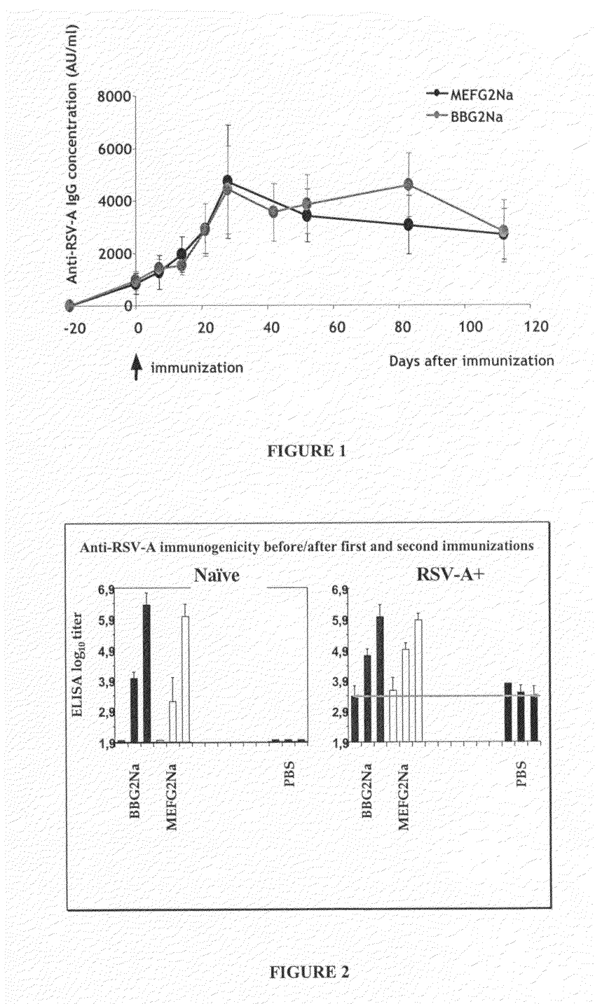

FIG. 2 also represents, in an additional representation, the anti-RSV-A IgG concentration in mice immunized with BBG2Na or MEFG2Na after 2 immunizations;

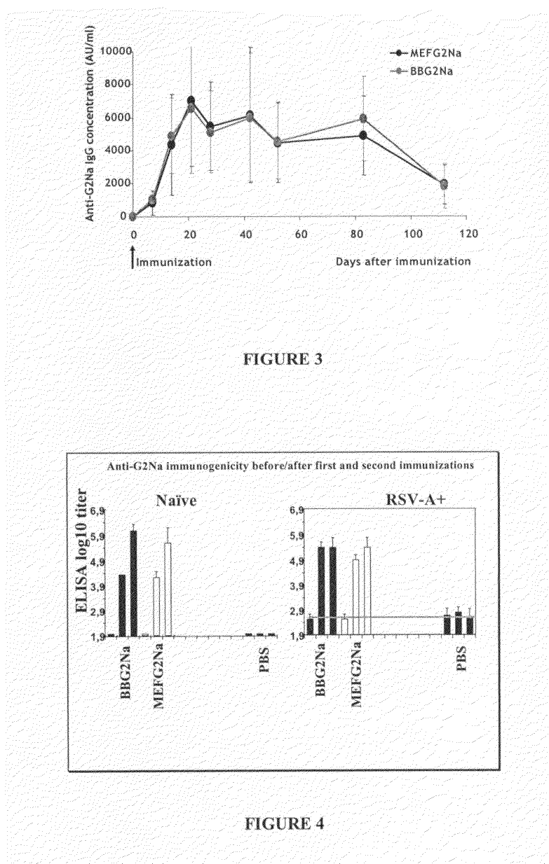

FIG. 3 represents the anti-G2Na IgG concentration in mice immunized with BBG2Na or MEFG2Na; and

FIG. 4 also represents, in an additional representation, the anti-G2Na IgG concentration in mice immunized with BBG2Na or MEFG2Na.

### EXAMPLE 1

### Comparison of in Vivo Activities Induced by the use of BB Carrier Protein or MEF Support Peptide

Eight-week-old IOPS female BALB/c mice are infected by nasal route with RSV-A Long strain (10⁵ pfu) at day 20. At day 0, after confirmation of RSV-A seroconversion, the mice receive a single intramuscular injection of 20 μg of BBG2Na (6 μg G2Na equivalent) adsorbed on Adju-Phos or 6 μg of MEFG2Na adsorbed on Adju-Phos. The concentrations of anti-RSV-A IgG (purified viral antigen) and anti-MEFG2Na are assayed by ELISA.

FIGS. 1 and 2 show that there is no significant difference between the concentrations of anti-RSV-A IgG triggered by 6 μg of MEFG2Na or 20 μg of BBG2Na at any point in the kinetics. The same is true for the concentration of anti-G2Na IgG (FIGS. 3 and 4).

### EXAMPLE 2

### Preparation of BBG2Na and MEFG2Na Complexes

### Preparation of BBG2Na:

BBG2Na protein is produced by using Escherichia coli RV308 as the host cell and a plasmid in which transcription of the gene of interest is under the control of the tryptophan promoter. The fermentation step is a batch method using a semi-defined synthetic culture medium and glycerol as a source of carbon and energy. Two culture steps are necessary to prepare the inoculum used in the production fermenter. In this fermenter, the microorganisms are grown to an optical density of 50 at 620 nm, then expression is induced by the addition of a tryptophan analogue (IAA). Growing continues until the partial pressure of O₂ in the fermenter rises suddenly, which indicates that the carbon source has been exhausted. At this stage the mean cell density is 40 g of dry cells/liter with a 9.5% expression rate, which is a productivity of 3.8 g of BBG2Na/liter of culture. The culture is cooled to +4 C and the microorganisms are recovered by centrifugation and frozen at −15 C to −25 C.

The extraction of BBG2Na requires solubilization of the defrosted pellet of microorganisms with a buffer containing guanidine, HCl and 1,4-dithiothreitol (DTT) to reduce disulfide bridges. Renaturation of the protein and oxidation of the disulfide bridges are obtained by dilution of the denatured suspension and shaking at ambient temperature overnight in an open reactor. The suspension containing the renatured protein is clarified by centrifugation and then filtered. Next, PEG 6000 is added to the filtrate and the resulting precipitate is recovered by centrifugation. The precipitate containing BBG2Na is solubilized again in a buffer containing urea. The extract obtained is filtered on a 0.22 μm support and stored at −15 C to −25 C.

Purification of BBG2Na from the defrosted extract consists of five steps: (1) cation exchange chromatography on a SP-Sepharose Fast Flow column; (2) hydrophobic interaction chromatography on a Macro-Prep Methyl column; (3) gel filtration on a Superdex S200 column; (4) anion exchange chromatography on a DEAE-Sepharose Fast Flow column; and finally, (5) a desalting step on a Sephadex G25 column. The solution of purified protein is filtered sterilely and distributed in sterile apyrogenic pouches.

### Preparation of MEFG2Na:

The MEFG2Na protein is produced by using Escherichia coli ICONE 200 as the host cell and a plasmid in which transcription of the gene of interest is under the control of the tryptophan promoter. E. coli ICONE 200 is a mutant of E. coli RV308 and was developed to improve control of expression during the growth phase. The fermentation step is a fed-batch method with a chemically defined culture medium and glycerol as a source of carbon and energy. Two culture steps are necessary to prepare the inoculum used in the production fermenter. In this fermenter, the microorganisms are grown to an optical density of 110 at 620 nm, then expression is induced by the addition of a tryptophan analogue (IAA). Growing continues until the partial pressure of O₂ in the fermenter rises suddenly, which indicates that the carbon source has been exhausted. At this stage the mean cell density is 56 g of dry cells/liter with a 5.4% expression rate, which is a productivity of 3 g of MEFG2Na/liter of culture. The culture is cooled to +4 C and the microorganisms are recovered by centrifugation and frozen at −15 C to −25 C.

Extraction of MEFG2Na requires solubilization of the defrosted pellet of microorganisms with a buffer containing guanidine and HCl. The suspension containing the renatured protein is clarified by centrifugation and then filtered. Since guanidine is incompatible with the subsequent purification step, a step of dialysis concentration on a polyethersulfone ultrafiltration support with a cut-off threshold of 10 kDa is used to carry out the buffer change. The extract obtained is filtered on a 0.22 μm support and then purified.

Purification of MEFG2Na is comprised of 3 steps: (1) cation exchange chromatography on a Fractogel EMD SE Hicap column; (2) gel filtration on a Superdex 75 Prep Grade column; and (3) anion exchange chromatography on a DEAE-Sepharose Fast Flow column. The bulk purified protein is filtered sterilely and distributed in sterile apyrogenic pouches.

### Expression Yields:

The expression data for MEFG2Na and BBG2Na are summarized in table 1 below.

TABLE 1
Quantity of MEFG2Na and BBG2Na protein obtained, expressed
in moles per 100 g of dry cells

| | Moles of protein per |
|---|---|
| | 100 g of dry cells |
| BBG2Na | 2.46 × 10⁻⁴ |
| MEFG2Na | 4.54 × 10⁻⁴ |

It appears that the expression rate of the MEFG2Na complex is approximately twice as great as the expression rate of the BBG2Na complex.

Although the present description, as well as the examples, are based only on the antigen G2Na, it should be understood that any immunogen can also be coupled to the support peptide according to the present invention.

1. A method for preparing an immunogenic complex in which an immunogen, antigen or hapten is associated with a support peptide to form the aforesaid immunogenic complex, wherein the aforesaid support peptide consists of a peptide of less than 10 amino acids comprising at least the peptide of sequence SEQ ID NO 2.

2. A method according to claim 1, wherein the aforesaid support peptide of less than 10 amino acids consists of the peptide coded by SEQ ID NO 2.

3. A method according to claim 1 or 2, wherein the aforesaid association consists of covalent coupling between the aforesaid peptide support and the aforesaid immunogen.

4. A method according to claim 3, wherein the aforesaid support peptide is coupled at the N-terminal end of the aforesaid immunogen when the aforesaid immunogen is a peptide.

5. A method according to claim 4, wherein the aforesaid covalent coupling is carried out by recombinant DNA technology.

6. A method according to claim 3 or 4, wherein the aforesaid covalent coupling is carried out by the chemical route.

7. A method according to one of the claims 1 to 6, wherein the immunogen is an antigen arising from bacteria, parasites and/or viruses.

8. A method according to claim 7, wherein the immunogen is a respiratory syncytial virus (RSV) surface protein or glycoprotein, a protein of a sequence having at least 80% identity with the sequence of the aforesaid RSV surface protein or a fragment of at least 10 consecutive amino acids of the aforesaid RSV surface protein, the aforesaid protein of a sequence having at least 80% identity or the aforesaid fragment being capable of inducing the production of specific antibodies directed against said protein or said fragment after the administration thereof in a mammal.

9. A method according to claim 8, wherein the immunogen is the human RSV type A or B G protein or the bovine RSV G protein, a protein of a sequence having at least 80% identity with the sequence of the aforesaid G protein or a fragment of the aforesaid G protein of at least 10 amino acids.

10. A method according to claim 9, wherein the immunogen is the polypeptide of the sequence between residues 130 and 230 of the RSV G protein, ends included, or of a sequence having at least 80% identity with the aforesaid sequence between residues 130 and 230, or a fragment of the aforesaid G protein of at least 10 amino acids.

11. A method according to claim 10, wherein the immunogen is the polypeptide of sequence SEQ ID NO 3.

12. An immunogenic complex obtained by the implementation of the method according to one of the claims 8 to 11.

13. An immunogenic complex comprising an immunogen, antigen or hapten, associated with a support peptide, wherein:
the aforesaid immunogen is associated, preferably coupled by a covalent link, with a support peptide of less than 10 amino acids comprising at least the peptide of sequence SEQ ID NO 2; and wherein
the immunogen is a syncytial respiratory virus (RSV) surface protein or glycoprotein, more particularly F or G, or is of a sequence having at least 80% identity with the sequence of the aforesaid RSV surface protein, capable of inducing the production of specific antibodies directed against said protein of a sequence having at least 80% identity after the administration thereof in a mammal.

14. A complex according to claim 13, wherein the aforesaid support peptide is the peptide of sequence SEQ ID NO 2.

15. A complex according to claim 12 or 13, wherein it is a MEFG2Na complex of sequence SEQ ID NO 4, or an immunogenic complex whose sequence presents in position 1 to 3 the sequence SEQ ID NO 2 followed by:
a sequence having at least 80% identity with sequence SEQ ID NO 3, preferably 85%, 90%, 95% or 98% identity with sequence SEQ ID NO 3.

16. A complex according to claim 15, of sequence SEQ ID NO 4.

17. A nucleic acid coding for an immunogenic complex according to one of the claims 13 to 16.

18. A nucleic acid according to claim 17 coding for the immunogenic complex of sequence SEQ ID NO 4.

19. A complex according to one of the claims 12 to 16, or a nucleic acid according to claim 17 or 18, used as a drug.

20. The use of an immunogenic complex according to one of the claims 12 to 16, or a nucleic acid according to claim 17 or 18, for the preparation of a pharmaceutical composition intended for the treatment or prevention of RSV-related respiratory infections.
